(19) United States
(12) Patent Application Publication
(10) Pub. No.: US 2019/0329223 A1
(43) Pub. Date: Oct. 31, 2019

(54) Sr-Ce-Yb-O Catalysts for Oxidative Coupling of Methane

(71) Applicant: Sabic Global Technologies, B.V., Bergen op Zoom (NL)

(72) Inventors: Wugeng LIANG, Sugar Land, TX (US); Vidya Sagar Reddy SARSANI, Sugar Land, TX (US); David WEST, Sugar Land, TX (US); Hector PEREZ, Sugar Land, TX (US); Yu-Lun FANG, Sugar Land, TX (US)

(21) Appl. No.: 16/347,328

(22) PCT Filed: Nov. 7, 2017
(86) PCT No.: PCT/US2017/060353

§ 371 Date: May 3, 2019

Related U.S. Application Data
(60) Provisional application No. 62/418,473, filed on Nov. 7, 2016.

(51) Int. Cl. B01J 23/10; B01J 37/03; C07C 11/04; C07C 2/84; B01J 23/00

(52) U.S. Cl. CPC B01J 23/10; B01J 37/031; B01J 23/002; C07C 2/84; C07C 11/04

(57) ABSTRACT

An oxidative coupling of methane (OCM) catalyst composition comprising (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof.

### CROSS REFERENCE TO RELATED APPLICATIONS

This application is a filing under 35 U.S.C. 371 of International Application No. PCT/US2017/060353 filed Nov. 7, 2017, entitled “Sr—Ce—Yb—O Catalysts for Oxidative Coupling of Methane” which claims priority to U.S. Provisional Application No. 62/418,473 filed Nov. 7, 2016, which applications are incorporated by reference herein in their entirety.

### TECHNICAL FIELD

The present disclosure relates to catalyst compositions for oxidative coupling of methane (OCM), more specifically catalyst compositions based on oxides of Sr, Ce and Yb for OCM and methods of making and using same.

### BACKGROUND

Hydrocarbons, and specifically olefins such as ethylene, are typically building blocks used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Oxidative coupling of the methane (OCM) has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically over a catalyst to form C₂H₄, water (H₂O) and heat.

Ethylene can be produced by OCM as represented by Equations (I) and (II):

2CH₄+O₂→C₂H₄+2H₂O ΔH=−67 kcal/mol (I)

2CH₄+½O₂→C₂H₆+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂O ΔH=−124 kcal/mol (III)

CH₄+2O₂→CO₂+2H₂O ΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored deep oxidation products.

Generally, in the OCM, CH₄ is first oxidatively converted into ethane (C₂H₆), and then into C₂H₄. CH₄ is activated heterogeneously on a catalyst surface, forming methyl free radicals (e.g., CH₃⁻), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with oxygen on the catalyst surface and/or in the gas phase, which produce (undesirable) carbon monoxide and carbon dioxide. Some of the best reported OCM outcomes encompass a ˜20% conversion of methane and −80% selectivity to desired C₂ hydrocarbons.

There are many catalyst systems developed for OCM processes, but such catalyst systems have many shortcomings. For example, conventional catalysts systems for OCM display catalyst performance problems, stemming from a need for high reaction temperatures. Thus, there is an ongoing need for the development of catalyst compositions for OCM processes.

### BRIEF SUMMARY

Disclosed herein is an oxidative coupling of methane (OCM) catalyst composition comprising (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof.

Also disclosed herein is a method of making an oxidative coupling of methane (OCM) catalyst composition comprising (a) forming a Sr—Ce—Yb—O precursor mixture, wherein the Sr—Ce—Yb—O precursor mixture comprises one or more compounds comprising a Sr cation, one or more compounds comprising a Ce cation, and one or more compounds comprising a Yb cation, and wherein the Sr—Ce—Yb—O precursor mixture is characterized by a molar ratio of Sr:(Ce+Yb) of about 1:1, and (b) calcining at least a portion of the Sr—Ce—Yb—O precursor mixture to form the OCM catalyst composition, wherein the OCM catalyst composition comprises Sr—Ce—Yb—O perovskite, and one or more oxides of a metal selected from the group consisting of Sr, Ce, and Yb.

Further disclosed herein is a method for producing olefins comprising (a) introducing a reactant mixture to a reactor comprising an oxidative coupling of methane (OCM) catalyst composition, wherein the reactant mixture comprises methane (CH₄) and oxygen (O₂), wherein the OCM catalyst composition comprises (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof, (b) allowing at least a portion of the reactant mixture to contact at least a portion of the OCM catalyst composition and react via an OCM reaction to form a product mixture comprising olefins, (c) recovering at least a portion of the product mixture from the reactor, and (d) recovering at least a portion of the olefins from the product mixture.

### BRIEF DESCRIPTION OF THE DRAWINGS

For a detailed description of the preferred embodiments of the disclosed methods, reference will now be made to the accompanying drawings in which:

FIG. 1 displays a graph of methane conversion in an oxidative coupling of methane (OCM) reaction as a function of temperature for catalysts prepared by various methods;

FIG. 2 displays a graph of oxygen conversion in an OCM reaction as a function of temperature for catalysts prepared by various methods;

FIG. 3 displays a graph of C₂₊ selectivity in an OCM reaction as a function of temperature for catalysts prepared by various methods; and

FIG. 4 displays an X-ray powder diffraction analysis for various catalysts.

### DETAILED DESCRIPTION

Disclosed herein are oxidative coupling of methane (OCM) catalyst compositions and methods of making and using same. In an aspect, an OCM catalyst composition can comprise (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof.

A method of making an oxidative coupling of methane (OCM) catalyst composition can generally comprise the steps of (a) forming a Sr—Ce—Yb—O precursor mixture, wherein the Sr—Ce—Yb—O precursor mixture comprises one or more compounds comprising a Sr cation, one or more compounds comprising a Ce cation, and one or more compounds comprising a Yb cation, and wherein the Sr—Ce—Yb—O precursor mixture is characterized by a molar ratio of Sr:(Ce+Yb) of about 1:1; and (b) calcining at least a portion of the Sr—Ce—Yb—O precursor mixture to form the OCM catalyst composition, wherein the OCM catalyst composition comprises Sr—Ce—Yb—O perovskite, and one or more oxides of a metal selected from the group consisting of Sr, Ce, and Yb. The one or more compounds comprising a Sr cation can comprise Sr nitrate, Sr oxide, Sr hydroxide, Sr chloride, Sr acetate, Sr carbonate, or combinations thereof; the one or more compounds comprising a Ce cation can comprise Ce nitrate, Ce oxide, Ce hydroxide, Ce chloride, Ce acetate, Ce carbonate, or combinations thereof; and the one or more compounds comprising a Yb cation can comprise Yb nitrate, Yb oxide, Yb hydroxide, Yb chloride, Yb acetate, Yb carbonate, or combinations thereof.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

In an aspect, a method for producing olefins can comprise introducing a reactant mixture to a reactor comprising an oxidative coupling of methane (OCM) catalyst composition to form a product mixture comprising olefins, wherein the reactant mixture comprises methane (CH₄) and oxygen (O₂), and wherein the OCM catalyst composition comprises (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof.

The reactant mixture can be a gaseous mixture. The reactant mixture can comprise a hydrocarbon or mixtures of hydrocarbons, and oxygen. In some aspects, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an aspect, the reactant mixture can comprise CH₄ and O₂.

The O₂ used in the reaction mixture can be oxygen gas (which may be obtained via a membrane separation process), technical oxygen (which may contain some air), air, oxygen enriched air, and the like, or combinations thereof.

The reactant mixture can further comprise a diluent. The diluent is inert with respect to the OCM reaction, e.g., the diluent does not participate in the OCM reaction. In an aspect, the diluent can comprise water, nitrogen, inert gases, and the like, or combinations thereof.

The diluent can provide for heat control of the OCM reaction, e.g., the diluent can act as a heat sink. Generally, an inert compound (e.g., a diluent) can absorb some of the heat produced in the exothermic OCM reaction, without degrading or participating in any reaction (OCM or other reaction), thereby providing for controlling a temperature inside the reactor.

The diluent can be present in the reactant mixture in an amount of from about 0.5% to about 80%, alternatively from about 5% to about 50%, or alternatively from about 10% to about 30%, based on the total volume of the reactant mixture.

A method for producing olefins can comprise introducing the reactant mixture to a reactor, wherein the reactor comprises the OCM catalyst composition. The reactor can comprise an adiabatic reactor, an autothermal reactor, an isothermal reactor, a tubular reactor, a cooled tubular reactor, a continuous flow reactor, a fixed bed reactor, a fluidized bed reactor, a moving bed reactor, and the like, or combinations thereof. In an aspect, the reactor can comprise a catalyst bed comprising the OCM catalyst composition.

The reaction mixture can be introduced to the reactor at a temperature of from about 150° C. to about 1,000° C., alternatively from about 225° C. to about 900° C., or alternatively from about 250° C. to about 800° C. As will be appreciated by one of skill in the art, and with the help of this disclosure, while the OCM reaction is exothermic, heat input is necessary for promoting the formation of methyl radicals from CH₄, as the C—H bonds of CH₄ are very stable, and the formation of methyl radicals from CH₄ is endothermic. In an aspect, the reaction mixture can be introduced to the reactor at a temperature effective to promote an OCM reaction.

The reactor can be characterized by a temperature of from about 400° C. to about 1,200° C., alternatively from about 500° C. to about 1,100° C., or alternatively from about 600° C. to about 1,000° C.

The reactor can be characterized by a pressure of from about ambient pressure (e.g., atmospheric pressure) to about 500 psig, alternatively from about ambient pressure to about 200 psig, or alternatively from about ambient pressure to about 150 psig. In an aspect, the method for producing olefins as disclosed herein can be carried out at ambient pressure.

The reactor can be characterized by a gas hourly space velocity (GHSV) of from about 500 h⁻¹ to about 10,000,000 h⁻¹, alternatively from about 500 h⁻¹ to about 1,000,000 h⁻¹, alternatively from about 500 h⁻¹ to about 500,000 h⁻¹, alternatively from about 1,000 h⁻¹ to about 500,000 h⁻¹, alternatively from about 1,500 h⁻¹ to about 500,000 h⁻¹, or alternatively from about 2,000 h⁻¹ to about 500,000 h⁻¹. Generally, the GHSV relates a reactant (e.g., reactant mixture) gas flow rate to a reactor volume. GHSV is usually measured at standard temperature and pressure.

The reactor can comprise an OCM catalyst composition comprising (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof. Generally, a perovskite refers to a compound having the same crystal structure as calcium titanate. For purposes of the disclosure herein, Sr—Ce—Yb—O perovskite of the OCM catalyst composition can be referred to as a “perovskite phase;” and the one or more oxides of the OCM catalyst composition can be referred to as an “oxide phase.” Without wishing to be limited by theory, the perovskite phase and the oxide phase have different physical and chemical properties, owing to having different crystal structures: the perovskite phase has a calcium titanate type of crystal structure, while the oxide phase has a crystal structure that is different than the calcium titanate type of crystal structure. The OCM catalyst composition can be regarded as a composite comprising the perovskite phase and the oxide phase, wherein the perovskite phase and the oxide phase can be interspersed. In some aspects, the OCM catalyst composition can comprise a continuous perovskite phase having a discontinuous oxide phase dispersed therein. In other aspects, the OCM catalyst composition can comprise a continuous oxide phase having a discontinuous perovskite phase dispersed therein. In yet other aspects, the OCM catalyst composition can comprise both a continuous perovskite phase and a continuous oxide phase, wherein the perovskite phase and the oxide phase contact each other. In still yet other aspects, the OCM catalyst composition can comprise regions of perovskite phase and regions of oxide phase, wherein at least a portion the regions of the perovskite phase contact at least a portion of the regions of the oxide phase.

As will be appreciated by one of skill in the art, and with the help of this disclosure, and without wishing to be limited by theory, the OCM reaction is a multi-step reaction, wherein each step of the OCM reaction could benefit from specific OCM catalytic properties. For example, and without wishing to be limited by theory, an OCM catalyst should exhibit some degree of basicity to abstract a hydrogen from CH₄ to form hydroxyl groups [OH] on the OCM catalyst surface, as well as methyl radicals (CH₃⁻). Further, and without wishing to be limited by theory, an OCM catalyst should exhibit oxidative properties for the OCM catalyst to convert the hydroxyl groups [OH] from the catalyst surface to water, which can allow for the OCM reaction to continue (e.g., propagate). Further, as will be appreciated by one of skill in the art, and with the help of this disclosure, and without wishing to be limited by theory, an OCM catalyst could also benefit from properties like oxygen ion conductivity and proton conductivity, which properties can be critical for the OCM reaction to proceed at a very high rate (e.g., its highest possible rate). Further, as will be appreciated by one of skill in the art, and with the help of this disclosure, and without wishing to be limited by theory, an OCM catalyst with a single phase might not provide all the necessary properties for an optimum OCM reaction (e.g., best OCM reaction outcome) at the best level, and as such conducting an optimum OCM reaction may require an OCM catalyst with tailored multi phases, wherein the various different phases can have optimum properties for various OCM reaction steps, and wherein the various different phases can provide synergistically for achieving the best performance for the OCM catalyst in an OCM reaction.

The Sr—Ce—Yb—O perovskite can be present in the OCM catalyst composition in an amount of from about 10.0 wt. % to about 90.0 wt. %, alternatively from about 15.0 wt. % to about 85.0 wt. %, or alternatively from about 20.0 wt. % to about 80.0 wt. %, based on the total weight of the OCM catalyst composition. The one or more oxides can be present in the OCM catalyst composition in an amount of from about 10.0 wt. % to about 90.0 wt. %, alternatively from about 15.0 wt. % to about 85.0 wt. %, or alternatively from about 20.0 wt. % to about 80.0 wt. %, based on the total weight of the OCM catalyst composition. As will be appreciated by one of skill in the art, and with the help of this disclosure, the amounts of each Sr—Ce—Yb—O perovskite and one or more oxides present in the OCM catalyst composition contribute to the distribution of the perovskite phase and the oxide phase within the OCM catalyst composition. Without wishing to be limited by theory, in addition to the amounts of each phase present in the OCM catalyst composition, the distribution of different phases in the catalyst composition is also important. For example, and without wishing to be limited by theory, a high activity phase (e.g., a phase containing CeO₂) could be dispersed and/or isolated in smaller fractions throughout the overall OCM catalyst composition in order to minimize and/or prevent deep oxidation reactions (e.g., CO₂ formation).

In an aspect, the one or more oxides of a metal selected from the group consisting of Sr, Ce, and Yb can comprise a single metal oxide, mixtures of single metal oxides, a mixed metal oxide, mixtures of mixed metal oxides, mixtures of both single metal oxides and mixed metal oxides, or combinations thereof.

Nonlimiting examples of the one or more oxides present in the OCM catalyst composition include CeO₂, CeYbO, Sr₂CeO₄, and the like, or combinations thereof. As will be appreciated by one of skill in the art, and with the help of this disclosure, a portion of the one or more oxides, in the presence of water, such as atmospheric moisture, can convert to hydroxides, and it is possible that the OCM catalyst composition will comprise some hydroxides, due to exposing the OCM catalyst composition comprising the one or more oxides to water (e.g., atmospheric moisture).

The single metal oxide comprises one metal cation selected from the group consisting of Sr, Ce, and Yb. A single metal oxide can be characterized by the general formula MₓO_{y}; wherein M is the metal cation selected from the group consisting of Sr, Ce, and Yb; and wherein x and y are integers from 1 to 7, alternatively from 1 to 5, or alternatively from 1 to 3. A single metal oxide contains one and only one metal cation. Nonlimiting examples of single metal oxides suitable for use in the OCM catalyst compositions of the present disclosure include CeO₂, Ce₂O₃, SrO, and Yb₂O₃.

In an aspect, mixtures of single metal oxides can comprise two or more different single metal oxides, wherein the two or more different single metal oxides have been mixed together to form the mixture of single metal oxides. Mixtures of single metal oxides can comprise two or more different single metal oxides, wherein each single metal oxide can be selected from the group consisting of CeO₂, Ce₂O₃, SrO, and Yb₂O₃. Nonlimiting examples of mixtures of single metal oxides suitable for use in the OCM catalyst compositions of the present disclosure include Yb₂O₃—CeO₂, Yb₂O₃—SrO, CeO₂—SrO, and the like, or combinations thereof.

The mixed metal oxide comprises two or more different metal cations, wherein each metal cation can be independently selected from the group consisting of Sr, Ce, and Yb. A mixed metal oxide can be characterized by the general formula Mₓ₁Mₓ₂O_{y}; wherein M¹ and M² are metal cations; wherein each of the M¹ and M² can be independently selected from the group consisting of Sr, Ce, and Yb; and wherein x1, x2 and y are integers from 1 to 15, alternatively from 1 to 10, or alternatively from 1 to 7. In some aspects, M¹ and M² can be cations of different chemical elements, for example M¹ can be a Ce cation and M² can be a Sr cation. In other aspects, M¹ and M² can be different cations of the same chemical element, wherein M¹ and M² can have different oxidation states. Nonlimiting examples of mixed metal oxides suitable for use in the OCM catalyst compositions of the present disclosure include CeYbO, Sr₂CeO₄, and the like, or combinations thereof.

In an aspect, mixtures of mixed metal oxides can comprise two or more different mixed metal oxides, wherein the two or more different mixed metal oxides have been mixed together to form the mixture of mixed metal oxides. Mixtures of mixed metal oxides can comprise two or more different mixed metal oxides, such as CeYbO and Sr₂CeO₄.

In an aspect, mixtures of single metal oxides and mixed metal oxides can comprise at least one single metal oxide and at least one mixed metal oxide, wherein the at least one single metal oxide and the at least one mixed metal oxide have been mixed together to form the mixture of single metal oxides and mixed metal oxides. Mixtures of single metal oxides and mixed metal oxides can comprise at least one single metal oxide and at least one mixed metal oxide, such as CeO₂ and Sr₂CeO₄; CeO₂, CeYbO, and Sr₂CeO₄; and the like; or combinations thereof.

In an aspect, the OCM catalyst composition can be characterized by the overall general formula SrCe₍₁₋ₓ₎YbₓO_{(3-x/2)}, wherein x can be from about 0.01 to about 0.99, alternatively from about 0.05 to about 0.95, or alternatively from about 0.1 to about 0.9. For purposes of the disclosure herein, the overall general formula accounts for both the perovskite phase and the oxide phase. As will be appreciated by one of skill in the art, and with the help of this disclosure, the overall general formula SrCe₍₁₋ₓ₎YbₓO_{(3-x/2)} further satisfies the condition of a molar ratio of Sr:(Ce+Yb) being about 1:1.

In some aspects, the OCM catalyst composition can be characterized by the overall general formula Sr_{1.0}Ce_{0.9}Yb_{0.1}O_{y}, wherein y balances the oxidation states. As will be appreciated by one of the skill in the art, and with the help of this disclosure, each of the Sr, Ce and Yb can have multiple oxidation states within the OCM catalyst composition, and as such y can have any suitable value that allows for the oxygen anions to balance all the cations. As will be appreciated by one of skill in the art, and with the help of this disclosure, the overall general formula Sr_{1.0}Ce_{0.9}Yb_{0.1}O_{y} further satisfies the condition of a molar ratio of Sr:(Ce+Yb) being about 1:1.

The OCM catalyst compositions suitable for use in the present disclosure can be supported OCM catalyst compositions and/or unsupported OCM catalyst compositions. In some aspects, the supported OCM catalyst compositions can comprise a support, wherein the support can be catalytically active (e.g., the support can catalyze an OCM reaction). In other aspects, the supported OCM catalyst compositions can comprise a support, wherein the support can be catalytically inactive (e.g., the support cannot catalyze an OCM reaction). In yet other aspects, the supported OCM catalyst compositions can comprise a catalytically active support and a catalytically inactive support. Nonlimiting examples of a support suitable for use in the present disclosure include MgO, Al₂O₃, SiO₂, ZrO₂, and the like, or combinations thereof. As will be appreciated by one of skill in the art, and with the help of this disclosure, the support can be purchased or can be prepared by using any suitable methodology, such as for example precipitation/co-precipitation, sol-gel techniques, templates/surface derivatized metal oxides synthesis, solid-state synthesis of mixed metal oxides, microemulsion techniques, solvothermal techniques, sonochemical techniques, combustion synthesis, etc.

In an aspect, the OCM catalyst composition can further comprise a support, wherein at least a portion of the OCM catalyst composition contacts, coats, is embedded in, is supported by, and/or is distributed throughout at least a portion of the support. In such aspect, the support can be in the form of powders, particles, pellets, monoliths, foams, honeycombs, and the like, or combinations thereof. Nonlimiting examples of support particle shapes include cylindrical, discoidal, spherical, tabular, ellipsoidal, equant, irregular, cubic, acicular, and the like, or combinations thereof.

In an aspect, the OCM catalyst composition can further comprise a porous support. As will be appreciated by one of skill in the art, and with the help of this disclosure, a porous material (e.g., support) can provide for an enhanced surface area of contact between the OCM catalyst composition and the reactant mixture, which in turn would result in a higher CH₄ conversion to CH₃⁻.

The OCM catalyst composition can be made by using any suitable methodology. In an aspect, a method of making an OCM catalyst composition can comprise a step of forming a Sr—Ce—Yb—O precursor mixture, wherein the Sr—Ce—Yb—O precursor mixture comprises one or more compounds comprising a Sr cation, one or more compounds comprising a Ce cation, and one or more compounds comprising a Yb cation, and wherein the Sr—Ce—Yb—O precursor mixture is characterized by a molar ratio of Sr:(Ce+Yb) of about 1:1.

The one or more compounds comprising a Sr cation comprises Sr nitrate, Sr oxide, Sr hydroxide, Sr chloride, Sr acetate, Sr carbonate, and the like, or combinations thereof. The one or more compounds comprising a Ce cation comprises Ce nitrate, Ce oxide, Ce hydroxide, Ce chloride, Ce acetate, Ce carbonate, and the like, or combinations thereof. The one or more compounds comprising a Yb cation comprises Yb nitrate, Yb oxide, Yb hydroxide, Yb chloride, Yb acetate, Yb carbonate, and the like, or combinations thereof.

In an aspect, the step of forming the Sr—Ce—Yb—O precursor mixture can comprise solubilizing the one or more compounds comprising a Sr cation, one or more compounds comprising a Ce cation, and one or more compounds comprising a Yb cation in an aqueous medium to form a Sr—Ce—Yb—O precursor aqueous solution. The aqueous medium can be water, or an aqueous solution. The Sr—Ce—Yb—O precursor aqueous solution can be formed by dissolving the one or more compounds comprising a Sr cation, one or more compounds comprising a Ce cation, one or more compounds comprising a Yb cation, or combinations thereof, in water or any suitable aqueous medium. As will be appreciated by one of skill in the art, and with the help of this disclosure, the one or more compounds comprising a Sr cation, one or more compounds comprising a Ce cation, and one or more compounds comprising a Yb cation can be dissolved in an aqueous medium in any suitable order. In some aspects, the one or more compounds comprising a Sr cation, one or more compounds comprising a Ce cation, and one or more compounds comprising a Yb cation can be first mixed together and then dissolved in an aqueous medium.

The Sr—Ce—Yb—O precursor aqueous solution can be dried to form the Sr—Ce—Yb—O precursor mixture. In an aspect, at least a portion of the Sr—Ce—Yb—O precursor aqueous solution can be dried at a temperature of equal to or greater than about 75° C., alternatively of equal to or greater than about 100° C., or alternatively of equal to or greater than about 125° C., to yield the Sr—Ce—Yb—O precursor mixture. The Sr—Ce—Yb—O precursor aqueous solution can be dried for a time period of equal to or greater than about 4 hours, alternatively equal to or greater than about 8 hours, or alternatively equal to or greater than about 12 hours.

In an aspect, a method of making an OCM catalyst composition can comprise a step of calcining at least a portion of the Sr—Ce—Yb—O precursor mixture to form the OCM catalyst composition, wherein the OCM catalyst composition comprises Sr—Ce—Yb—O perovskite, and one or more oxides of a metal selected from the group consisting of Sr, Ce, and Yb. The Sr—Ce—Yb—O precursor mixture can be calcined at a temperature of equal to or greater than about 650° C., alternatively equal to or greater than about 800° C., or alternatively equal to or greater than about 900° C., to yield the OCM catalyst composition. The Sr—Ce—Yb—O precursor mixture can be calcined for a time period of equal to or greater than about 2 hours, alternatively equal to or greater than about 4 hours, or alternatively equal to or greater than about 6 hours.

In some aspects, at least a portion of the Sr—Ce—Yb—O precursor mixture can be calcined in an oxidizing atmosphere (e.g., in an atmosphere comprising oxygen, for example in air) to form the OCM catalyst composition. Without wishing to be limited by theory, the oxygen in the Sr—Ce—Yb—O perovskite and/or one or more oxides of a metal selected from the group consisting of Sr, Ce, and Yb can originate in the oxidizing atmosphere used for calcining the Sr—Ce—Yb—O precursor mixture. Further, without wishing to be limited by theory, the oxygen in the Sr—Ce—Yb—O perovskite and/or one or more oxides of a metal selected from the group consisting of Sr, Ce, and Yb can originate in the one or more compounds comprising a Sr cation, one or more compounds comprising a Ce cation, and one or more compounds comprising a Yb cation, provided that at least one of these compounds comprises oxygen in its formula, as is the case with nitrates, oxides, hydroxides, acetates, carbonates, etc.

In some aspects, the method of making an OCM catalyst composition can further comprise contacting the OCM catalyst composition with a support to yield a supported catalyst (e.g., an OCM supported catalyst, an OCM supported catalyst composition, etc.).

In other aspects, the method of making an OCM catalyst composition can comprise forming the OCM catalyst composition in the presence of the support, such that the resulting OCM catalyst composition (after the calcining step) comprises the support.

In an aspect, a method for producing olefins can comprise allowing at least a portion of the reactant mixture to contact at least a portion of the OCM catalyst composition and react via an OCM reaction to form a product mixture comprising olefins.

The product mixture comprises coupling products, partial oxidation products (e.g., partial conversion products, such as CO, H₂, CO₂), and unreacted methane. The coupling products can comprise olefins (e.g., alkenes, characterized by a general formula CₙH₂ₙ) and paraffins (e.g., alkanes, characterized by a general formula CₙH₂ₙ₊₂).

The product mixture can comprise C₂₊ hydrocarbons, wherein the C₂₊ hydrocarbons can comprise C₂ hydrocarbons and C₃ hydrocarbons. In an aspect, the C₂₊ hydrocarbons can further comprise C₄ hydrocarbons (C₄s), such as for example butane, iso-butane, n-butane, butylene, etc. The C₂ hydrocarbons can comprise ethylene (C₂H₄) and ethane (C₂H₆). The C₂ hydrocarbons can further comprise acetylene (C₂H₂). The C₃ hydrocarbons can comprise propylene (C₃H₆) and propane (C₃H₈).

Reactant conversions (e.g., methane conversion, oxygen conversion, etc.) and selectivities to certain products (e.g., selectivity to C₂₊ hydrocarbons, selectivity to C₂ hydrocarbons, selectivity to ethylene, etc.) can be calculated as disclosed in more detail in the Examples section, for example such as described in equations (1)-(3).

In an aspect, equal to or greater than about 10 mol %, alternatively equal to or greater than about 30 mol %, or alternatively equal to or greater than about 50 mol % of the methane in the reactant mixture can be converted to C₂₊ hydrocarbons.

In an aspect, the OCM catalyst composition can be characterized by a C₂₊ selectivity that is increased by equal to or greater than about 5%, alternatively equal to or greater than about 10%, or alternatively equal to or greater than about 20%, when compared to a C₂₊ selectivity of an otherwise similar OCM catalyst composition comprising, consisting of, or consisting essentially of Sr—Ce—Yb—O perovskite without the one or more oxides. Generally, a selectivity to a certain product refers to the amount of that particular product formed divided by the total amount of products formed.

In an aspect, the OCM catalyst composition can be characterized by a C₂₊ productivity that is increased by equal to or greater than about 50%, alternatively equal to or greater than about 100%, or alternatively equal to or greater than about 200%, when compared to a C₂₊ productivity of an otherwise similar OCM catalyst composition comprising, consisting of, or consisting essentially of Sr—Ce—Yb—O perovskite without the one or more oxides. The productivity with respect to C₂₊ hydrocarbons refers to the amount of C₂₊ hydrocarbons recovered from the product mixture (which can be expressed as volume, mass, moles, etc.) per unit of time (e.g., hours, minutes, seconds, etc.) per amount of catalyst used (e.g., g, kg, lb, etc.). The productivity with respect to a certain catalyst is a measure of effectiveness for that particular catalyst.

In an aspect, a method for producing olefins can comprise recovering at least a portion of the product mixture from the reactor, wherein the product mixture can be collected as an outlet gas mixture from the reactor. In an aspect, a method for producing olefins can comprise recovering at least a portion of the C₂ hydrocarbons from the product mixture. The product mixture can comprise C₂₊ hydrocarbons (including olefins), unreacted methane, and optionally a diluent. The water produced from the OCM reaction and the water used as a diluent (if water diluent is used) can be separated from the product mixture prior to separating any of the other product mixture components. For example, by cooling down the product mixture to a temperature where the water condenses (e.g., below 100° C. at ambient pressure), the water can be removed from the product mixture, by using a flash chamber for example.

In an aspect, at least a portion of the C₂₊ hydrocarbons can be separated (e.g., recovered) from the product mixture to yield recovered C₂₊ hydrocarbons. The C₂₊ hydrocarbons can be separated from the product mixture by using any suitable separation technique. In an aspect, at least a portion of the C₂₊ hydrocarbons can be separated from the product mixture by distillation (e.g., cryogenic distillation).

In an aspect, at least a portion of the recovered C₂₊ hydrocarbons can be used for ethylene production. In some aspects, at least a portion of ethylene can be separated from the product mixture (e.g., from the C₂₊ hydrocarbons, from the recovered C₂₊ hydrocarbons) to yield recovered ethylene and recovered hydrocarbons, by using any suitable separation technique (e.g., distillation). In other aspects, at least a portion of the recovered hydrocarbons (e.g., recovered C₂₊ hydrocarbons after olefin separation, such as separation of C₂H₄ and C₃H₆) can be converted to ethylene, for example by a conventional steam cracking process.

A method for producing olefins can comprise recovering at least a portion of the olefins from the product mixture. In an aspect, at least a portion of the olefins can be separated from the product mixture by distillation (e.g., cryogenic distillation). As will be appreciated by one of skill in the art, and with the help of this disclosure, the olefins are generally individually separated from their paraffin counterparts by distillation (e.g., cryogenic distillation). For example ethylene can be separated from ethane by distillation (e.g., cryogenic distillation). As another example, propylene can be separated from propane by distillation (e.g., cryogenic distillation).

In an aspect, at least a portion of the unreacted methane can be separated from the product mixture to yield recovered methane. Methane can be separated from the product mixture by using any suitable separation technique, such as for example distillation (e.g., cryogenic distillation). At least a portion of the recovered methane can be recycled to the reactant mixture.

In an aspect, an OCM catalyst composition can comprise (i) from about 15.0 wt. % to about 85.0 wt. % Sr—Ce—Yb—O perovskite (e.g., SrCe_{0.95}Yb_{0.05}O_{2.975} with perovskite structure); and (ii) from about 15.0 wt. % to about 85.0 wt. % one or more oxides of a metal selected from the group consisting of Sr, Ce, and Yb, wherein the one or more oxides comprises a single metal oxide, mixtures of single metal oxides, a mixed metal oxide, mixtures of mixed metal oxides, mixtures of single metal oxides and mixed metal oxides, and the like, or combinations thereof. In such aspect, the OCM catalyst composition can be characterized by the overall general formula Sr_{1.0}Ce_{0.9}Yb_{0.1}O_{y}, wherein y balances the oxidation states.

In an aspect, an OCM catalyst composition can comprise (i) from about 20.0 wt. % to about 80.0 wt. % Sr—Ce—Yb—O perovskite (e.g., SrCeYbO₃ with perovskite structure); and (ii) from about 20.0 wt. % to about 80.0 wt. % one or more oxides of a metal selected from the group consisting of Sr, Ce, and Yb, wherein the one or more oxides comprises CeO₂, CeYbO, Sr₂CeO₄, and the like, or combinations thereof. In such aspect, the OCM catalyst composition can be characterized by the overall general formula Sr_{1.0}Ce_{0.9}Yb_{0.1}O_{y}, wherein y balances the oxidation states.

In an aspect, a method of making an OCM catalyst composition can comprise the steps of (a) forming a Sr—Ce—Yb—O precursor aqueous solution comprising Sr nitrate, Ce nitrate, and Yb nitrate, wherein the Sr—Ce—Yb—O precursor aqueous solution is characterized by a molar ratio of Sr:(Ce+Yb) of about 1:1; (b) drying at least a portion of the Sr—Ce—Yb—O precursor aqueous solution at a temperature of about 125° C. for about 12-18 h to form a Sr—Ce—Yb—O precursor mixture; and (c) calcining at least a portion of the Sr—Ce—Yb—O precursor mixture at a temperature of about 900° C. for about 4-8 h, for example in an oxidizing atmosphere, to form the OCM catalyst composition, wherein the OCM catalyst composition comprises a Sr—Ce—Yb—O perovskite, and one or more oxides of a metal selected from the group consisting of Sr, Ce, and Yb.

In an aspect, a method for producing ethylene can comprise the steps of (a) introducing a reactant mixture to a reactor comprising an oxidative coupling of methane (OCM) catalyst composition, wherein the reactant mixture comprises methane (CH₄) and oxygen (O₂), wherein the OCM catalyst composition comprises (i) from about 20.0 wt. % to about 80.0 wt. % Sr—Ce—Yb—O perovskite (e.g., SrCeYbO₃ with perovskite structure); and (ii) from about 20.0 wt. % to about 80.0 wt. % one or more oxides of a metal selected from the group consisting of Sr, Ce, and Yb, wherein the one or more oxides comprises a single metal oxide, mixtures of single metal oxides, a mixed metal oxide, mixtures of mixed metal oxides, mixtures of single metal oxides and mixed metal oxides, and the like, or combinations thereof; (b) allowing at least a portion of the reactant mixture to contact at least a portion of the OCM catalyst composition and react via an OCM reaction to form a product mixture comprising olefins, wherein the olefins comprise ethylene; (c) recovering at least a portion of the product mixture from the reactor; and (d) recovering at least a portion of the ethylene from the product mixture.

In an aspect, the OCM catalyst compositions comprising (i) Sr—Ce—Yb—O perovskite (e.g., SrCeYbO₃ with perovskite structure); and (ii) one or more oxides of a metal selected from the group consisting of Sr, Ce, and Yb, and methods of making and using same, as disclosed herein can advantageously display improvements in one or more composition characteristics when compared to an otherwise similar OCM catalyst composition comprising, consisting of, or consisting essentially of Sr—Ce—Yb—O perovskite without the one or more oxides.

The OCM catalyst compositions comprising (i) Sr—Ce—Yb—O perovskite (e.g., SrCeYbO₃ with perovskite structure); and (ii) one or more oxides of a metal selected from the group consisting of Sr, Ce, and Yb, can display improved selectivity and productivity when compared to the selectivity and productivity of an otherwise similar OCM catalyst composition comprising, consisting of, or consisting essentially of Sr—Ce—Yb—O perovskite without the one or more oxides. As will be appreciated by one of skill in the art, and with the help of this disclosure, having a high productivity catalyst, such as the OCM catalyst compositions disclosed herein (OCM catalyst compositions comprising (i) Sr—Ce—Yb—O perovskite; and (ii) one or more oxides of a metal selected from the group consisting of Sr, Ce, and Yb), to achieve the same production as with a conventional OCM catalyst (similar OCM catalyst composition comprising, consisting of, or consisting essentially of Sr—Ce—Yb—O perovskite without the one or more oxides), the reactor size can be much smaller, and consequently the production cost can be reduced. Additional advantages of the OCM catalyst compositions comprising (i) Sr—Ce—Yb—O perovskite (e.g., SrCeYbO₃ with perovskite structure); and (ii) one or more oxides of a metal selected from the group consisting of Sr, Ce, and Yb, and methods of making and using same, as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

OCM catalyst compositions comprising Sr_{1.0}Ce_{0.9}Yb_{0.1}Oₓ were prepared as follows.

4.23 g of Sr(NO₃)₂, 7.82 g of Ce(NO₃)₃×6H₂O and 0.90 g of Yb(NO₃)₃×5H₂O were mixed with 25 ml deionized (DI) water to provide a mixture, which mixture was further agitated until all solids were dissolved and a clear solution was obtained. The obtained clear solution was dried at 125° C. overnight to produce a dried Sr—Ce—Yb—O precursor mixture.

The dried Sr—Ce—Yb—O precursor mixture was calcined at 900° C. under air flow for 6 hours to produce the Sr_{1.0}Ce_{0.9}Yb_{0.1}Oₓ (900° C. calcination) catalyst.

The dried Sr—Ce—Yb—O precursor mixture was calcined at 1,100° C. under air flow for 6 hours to produce the Sr_{1.0}Ce_{0.9}Yb_{0.1}Oₓ (1,100° C. calcination) catalyst.

The dried Sr—Ce—Yb—O precursor mixture was calcined at 1,300° C. under air flow for 6 hours to produce the Sr_{1.0}Ce_{0.9}Yb_{0.1}Oₓ (1,300° C. calcination) catalyst.

### Example 2

The performance of the OCM catalyst compositions comprising Sr_{1.0}Ce_{0.9}Yb_{0.1}Oₓ prepared as described in Example 1 was investigated.

Oxidative coupling of methane (OCM) reactions were conducted by using catalysts prepared as described in Example 1 as follows. A mixture of methane and oxygen along with an internal standard, an inert gas (neon) were fed to a quartz reactor with an internal diameter (I.D.) of 2.3 mm heated by traditional clamshell furnace. A catalyst (e.g., catalyst bed) loading was 20 mg, and total flow rate of reactants was 40 standard cubic centimeters per minute (sccm). The reactor was first heated to a desired temperature under an inert gas flow and then a desired gas mixture was fed to the reactor. All OCM reactions were conducted at a methane to oxygen (CH₄:O₂) molar ratio of 7.4.

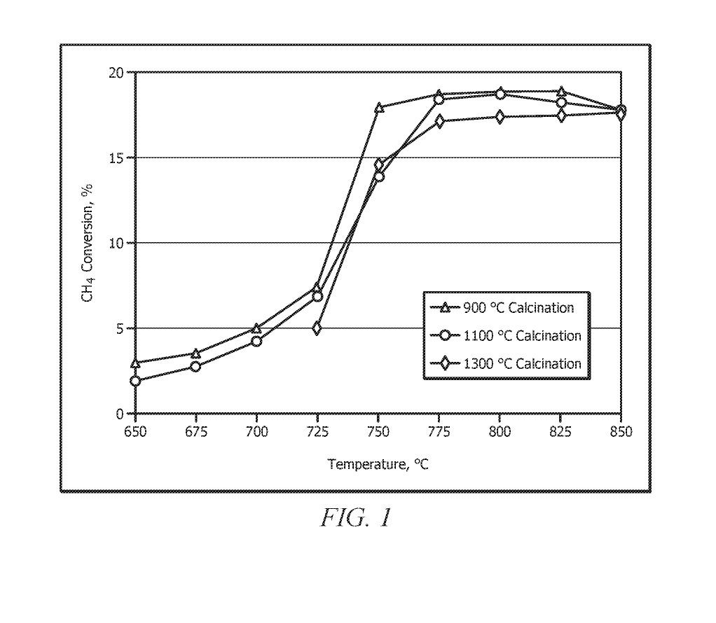

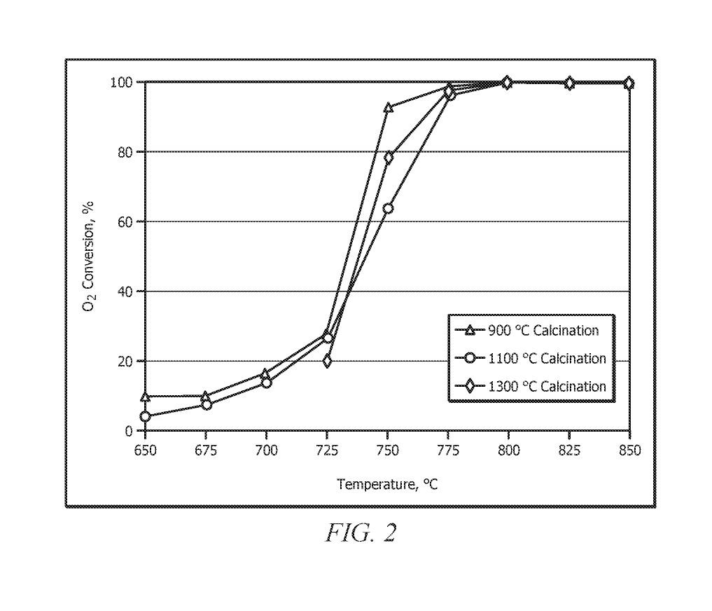

The performance of the three types of catalysts is illustrated in FIGS. 1-3. By comparing CH₄ and O₂ conversions at different temperatures, it can be seen that after a higher calcination temperature used for preparing the OCM catalyst composition, the catalyst activity is reduced, and a higher temperature is needed to get the same conversions.

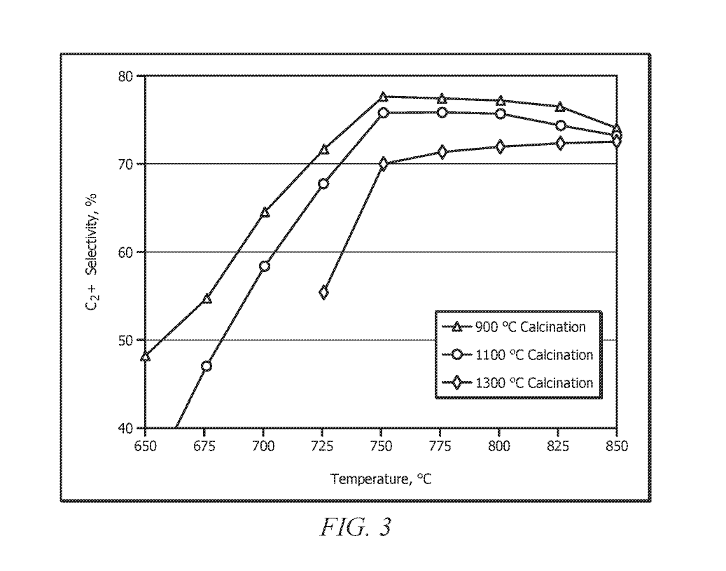

The OCM catalysts prepared by calcination at higher temperatures (1,100° C. and 1,300° C. calcination) show lower selectivity as well, as shown in FIG. 3.

Methane conversion was calculated according to equation (1). Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a % conversion based on moles converted. For example, the methane conversion can be calculated by using equation (1):

$${CH}_{4} conversion = \frac{{C}_{{CH}_{4}}^{i n} - {C}_{{CH}_{4}}^{out}}{{C}_{{CH}_{4}}^{i n}} \times 100 \% \qquad ( 1 )$$

wherein C_{CH}_{₄}ⁱⁿ=number of moles of C from CH₄ that entered the reactor as part of the reactant mixture; and C_{CH}_{₄}^{out}=number of moles of C from CH₄ that was recovered from the reactor as part of CH₄ the product mixture.

The oxygen conversion can be calculated by using equation (2):

$${O}_{2} conversion = \frac{{O}_{2}^{i n} - {O}_{2}^{out}}{{O}_{2}^{i n}} \times 100 \% \qquad ( 2 )$$

wherein O₂ⁱⁿ=number of moles of O₂ that entered the reactor as part of the reactant mixture; and O₂^{out}=number of moles of O₂ that was recovered from the reactor as part of the product mixture.

Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a Cₓ selectivity (e.g., C₂ selectivity, C₂₊ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{C3H8}=number of moles of C from CH₄ that were converted into C₃H; C_{C4s}=number of moles of C from CH₄ that were converted into C₄ hydrocarbons (C₄s); C_{C02}=number of moles of C from CH₄ that were converted into CO₂; C_{CO}=number of moles of C from CH₄ that were converted into CO; etc.

A C₂₊ selectivity (e.g., selectivity to C₂₊ hydrocarbons) refers to how much C₂H₄, C₃H₆, C₂H₂, C₂H₆, C₃H₈, and C₄s were formed divided by the total products formed, including C₂H₄, C₃H₆, C₂H₂, C₂H₆, C₃H₈, C₄s, CO₂ and CO. For example, the C₂₊ selectivity can be calculated by using equation (3):

$${C}_{2 +} selectivity = \frac{2 {C}_{{C}_{2} {H}_{4}} + 2 {C}_{{C}_{2} {H}_{6}} + 2 {C}_{{C}_{2} {H}_{2}} + 3 {C}_{{C}_{3} {H}_{6}} + 3 {C}_{{C}_{3} {H}_{8}} + 4 {C}_{{C}_{4 s}}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4 s}} + {C}_{{CO}_{2}} + {C}_{CO}\end{matrix}} \times 100 \% \qquad ( 3 )$$

Further, a C₂₊ yield can be calculated as the product of C₂₊ selectivity and methane conversion, for example by using equation (4):

C₂₊ yield=methane conversion×C₂₊ selectivity (4)

For example, if a certain OCM reaction/process is characterized by a 50% methane conversion, and by a 50% C₂₊ selectivity, the resulting C₂₊ yield can be calculated as being 25% (=50%×50%).

As will be appreciated by one of skill in the art, if a specific product and/or hydrocarbon product is not produced in a certain OCM reaction/process, then the corresponding C_{Cx} is 0, and the term is simply removed from selectivity calculations.

The performance differences between the three types of catalysts are also demonstrated in Table 1. Run #1 corresponds to the Sr_{1.0}Ce_{0.9}Yb_{0.1}Oₓ (900° C. calcination) catalyst; run #2 corresponds to the Sr_{1.0}Ce_{0.9}Yb_{0.1}Oₓ (1,100° C. calcination) catalyst; and run #3 corresponds to the Sr_{1.0}Ce_{0.9}Yb_{0.1}Oₓ (1,300° C. calcination) catalyst. The data in Table 1 were collected as described for FIGS. 1-3, except for the flow rate, which was 60 sccm for the data in Table 1.

TABLE 1

| | Reaction | CH₄ | O₂ | C₂₊ | CO | CO₂ | C₂₊ |
|---|---|---|---|---|---|---|---|
| | Temperature | Conversion | Conversion | Selectivity | Selectivity | Selectivity | Yield |
| | (° C.) | (%) | (%) | (%) | (%) | (%) | (%) |
| Run #1 | 725 | 19.8 | 97.3 | 79.8 | 1.5 | 18.6 | 15.8 |
| Run #2 | 725 | 17.2 | 96.3 | 74.8 | 3.2 | 22.1 | 12.9 |
| Run #3 | 750 | 17.4 | 100.0 | 73.8 | 3.7 | 22.5 | 12.8 |

The data in Table 1 are optimized yield results obtained at a methane to oxygen ratio of 7.4. Yield of run#1 acquired for the Sr_{1.0}Ce_{0.9}Yb_{0.1}Oₓ (900° C. calcination) catalyst was about 20% more than the yield of run#2 acquired for the Sr_{1.0}Ce_{0.9}Yb_{0.1}Oₓ (1100° C. calcination) catalyst and run#3 acquired for the Sr_{1.0}Ce_{0.9}Yb_{0.1}Oₓ (1300° C. calcination) catalyst. The better yield of run#1 is a result of its better C₂₊ selectivity and higher methane conversion. It can also be seen that a lower reaction temperature was used for run#1 to achieve these results, indicating better activity for the catalyst used in run #1 (Sr_{1.0}Ce_{0.9}Yb_{0.1}Oₓ (900° C. calcination) catalyst). However, the catalyst performance could be further enhanced by optimizing the combination of perovskite phase and other oxide phases in the catalyst to provide for the required properties necessary for enhancing catalyst performance.

### Example 3

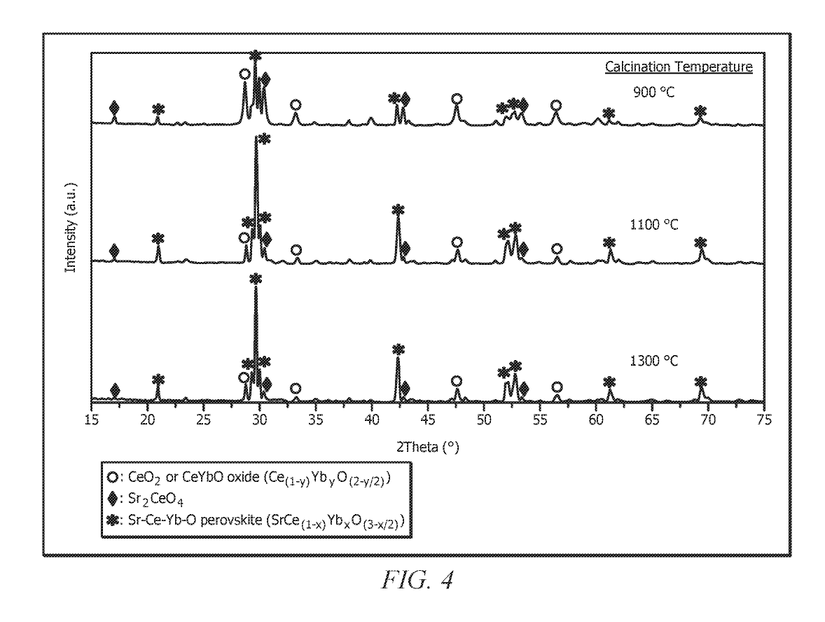

The OCM catalyst compositions comprising Sr_{1.0}Ce_{0.9}Yb_{0.1}Oₓ prepared as described in Example 1 were further investigated by X-ray powder diffraction (XRD), and the data are shown in FIG. 4. XRD measurements were performed with PANalytical X′Pert (X-ray source: Cu K_{α1}, wavelength: 1.54 Å, scan range: 2 Theta=10°˜90°, step size: 0.02°). Estimated weight contents of different phases were determined by normalized reference-intensity-ratio (RIR) method. The phase content for each catalyst composition is shown in Table 2.

TABLE 2

| | Sr—Ce—Yb—O with | | |
|---|---|---|---|
| Calcination | perovskite structure | CeO₂ or CeYbO oxide | |
| Temperature | (SrCe₍₁₋ₓ₎YbₓO_{(3-x/2)}) | (Ce_{(1-y)}Yb_{y}O_{(2-y/2)}) | Sr₂CeO₄ |
| (° C.) | (%) | (%) | (%) |
| 900 | 31 | 15 | 54 |
| 1,100 | 74 | 4 | 22 |
| 1,300 | 91 | 9 | ~0 |

Note: As will be appreciated by one of skill in the art, and with the help of this disclosure, for the formulas in Table 2, x can be 0 in some instances, wherein the perovskite phase comprises a Sr—Ce—O oxide with perovskite structure. However, when x=0, y cannot be 0 at the same time to provide for the Ce_{(1-y)}Yb_{y}O_{(2-y/2)} being CeO₂; such that the OCM catalyst composition contains Yb at all times. Further, as will be appreciated by one of skill in the art, and with the help of this disclosure, in some instances, both x and y can have very small values, for example less than 0.1.

XRD data indicate that in addition to the perovskite phase (e.g., SrCe₍₁₋ₓ₎YbₓO_{(3-x/2)} with perovskite structure) in the catalysts, there are other oxides, such as CeO₂ and/or CeYbO (Ce_{(1-y)}Yb_{y}O_{(2-y/2)}), and Sr₂CeO₄ existing in the catalysts. As will be appreciated by one of skill in the art, and with the help of this disclosure, when y has very small values, for example less than about 0.1, XRD cannot distinguish between CeO₂ and a mixed oxide having both Ce and Yb according to formula Ce_{(1-y)}Yb_{y}O_{(2y/2)}, and as such it is possible that the analyzed composition has CeO₂; a mixed oxide having both Ce and Yb according to formula Ce_{(1-y)}Yb_{y}O_{(2y/2)}; or both CeO₂ and a mixed oxide having both Ce and Yb according to formula Ce_{(1-y)}Yb_{y}O_{(2y/2)}.

When preparing the OCM catalyst compositions, as the calcination temperature was increased from 900° C. to 1,100° C. and 1,300° C., the amount of perovskite phase in the catalyst composition increased as well, with decreasing the amount of the non-perovskite phase oxides. Although the higher calcination temperature increases the perovskite structure content in the catalyst composition, catalyst performance data as shown above in Table 1 indicate that an increased amount of perovskite lowers catalyst activity and selectivity. Therefore, in addition to perovskite, a certain amount of other oxides in the catalyst compositions, such as CeO₂ and/or CeYbO, and Sr₂CeO₄ oxides, could yield a better performing OCM catalyst.

### Example 4

The performance of OCM catalyst compositions comprising the Sr_{1.0}Ce_{0.9}Yb_{0.1}Oₓ (900° C. calcination) catalyst prepared as described in Example 1 were compared to data available in the literature: J. Chem. Soc., Chem. Commun., 1987, p. 1639 (Literature (1); and J. Chem. Soc. Faraday Trans., 91 (1995), p. 1179 (Literature (2)), each of which is incorporated by reference herein in its entirety. The results of the comparison are displayed in Table 3.

TABLE 3

| | | | | CH₄ | C₂₊ | C₂₊ |
|---|---|---|---|---|---|---|
| | Temperature | Catalyst | CH₄ flowrate | Conversion | Selectivity | Productivity |
| Catalyst | (° C.) | loading | (ml/min) | (%) | (%) | (cc/min/g) |
| Sr_{1.0}Ce_{0.9}Yb_{0.1}Oₓ | 725 | 20 mg | 43.6 | 20.1 | 79.9 | 348.4 |
| (900° C. calcination) | | | | | | |
| catalyst | | | | | | |
| Literature (1) | 750 | 600 mg | 3.3 | 52.6 | 60.1 | 1.74 |
| Literature (2) | 775 | 500 mg | 40.0 | 20.0 | 60.0 | 9.6 |

The C₂₊ productivity of each catalyst was calculated as the C₂₊ formed (cc/min) over the same amount of the catalyst. The productivity of the Sr_{1.0}Ce_{0.9}Yb_{0.1}Oₓ (900° C. calcination) catalyst prepared as described in Example 1 was significantly higher than that of the catalysts from the literature. The literature catalysts are Sr—Ce—Yb—O catalysts with pure perovskite structure, and as such the data in Table 3 indicate the superior performance from the catalysts disclosed herein comprising other oxides in addition to the perovskite oxides. The data in Table 3 further confirm that a catalyst having tailored multi phases with required properties will perform better than a catalyst having a single phase alone.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### ADDITIONAL DISCLOSURE

A first aspect, which is an oxidative coupling of methane (OCM) catalyst composition comprising (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof.

A second aspect, which is the OCM catalyst composition of the first aspect, wherein the one or more oxides comprise CeO₂, CeYbO, Sr₂CeO₄, or combinations thereof.

A third aspect, which is the OCM catalyst composition of any one of the first and the second aspects, wherein the single metal oxide comprises one metal cation selected from the group consisting of Sr, Ce, and Yb.

A fourth aspect, which is the OCM catalyst composition of any one of the first through the third aspects, wherein the single metal oxide comprises CeO₂.

A fifth aspect, which is the OCM catalyst composition of any one of the first through the fourth aspects, wherein the mixed metal oxide comprises two or more different metal cations, wherein each metal cation can be independently selected from the group consisting of Sr, Ce, and Yb.

A sixth aspect, which is the OCM catalyst composition of any one of the first through the fifth aspects, wherein the mixed metal oxide comprises CeYbO, Sr₂CeO₄, or both CeYbO and Sr₂CeO₄.

A seventh aspect, which is the OCM catalyst composition of any one of the first through the sixth aspects having the overall general formula SrCe₍₁₋ₓ₎YbₓO_{(3-x/2)}, wherein x is from about 0.01 to about 0.99.

An eighth aspect, which is the OCM catalyst composition of any one of the first through the seventh aspects having the overall general formula Sr_{1.0}Ce_{0.9}Yb_{0.1}O_{y}, wherein y balances the oxidation states.

A ninth aspect, which is the OCM catalyst composition of any one of the first through the eighth aspects comprising (i) from about 10.0 wt. % to about 90.0 wt. % Sr—Ce—Yb—O perovskite; and (ii) from about 10.0 wt. % to about 90.0 wt. % one or more oxides.

A tenth aspect, which is the OCM catalyst composition of any one of the first through the ninth aspects further comprising a support, wherein at least a portion of the OCM catalyst composition contacts, coats, is embedded in, is supported by, and/or is distributed throughout at least a portion of the support; wherein the support comprises MgO, Al₂O₃, SiO₂, ZrO₂, or combinations thereof; and wherein the support is in the form of particles, pellets, monoliths, foams, honeycombs, or combinations thereof.

An eleventh aspect, which is the OCM catalyst composition of any one of the first through the tenth aspects, wherein the OCM catalyst composition is characterized by a C₂₊ selectivity that is increased by equal to or greater than about 5%, when compared to a C₂₊ selectivity of an otherwise similar OCM catalyst composition comprising Sr—Ce—Yb—O perovskite without the one or more oxides.

A twelfth aspect, which is the OCM catalyst composition of any one of the first through the eleventh aspects, wherein the OCM catalyst composition is characterized by a C₂₊ productivity that is increased by equal to or greater than about 50%, when compared to a C₂₊ productivity of an otherwise similar OCM catalyst composition comprising Sr—Ce—Yb—O perovskite without the one or more oxides.

A thirteenth aspect, which is a method of making an oxidative coupling of methane (OCM) catalyst composition comprising (a) forming a Sr—Ce—Yb—O precursor mixture, wherein the Sr—Ce—Yb—O precursor mixture comprises one or more compounds comprising a Sr cation, one or more compounds comprising a Ce cation, and one or more compounds comprising a Yb cation, and wherein the Sr—Ce—Yb—O precursor mixture is characterized by a molar ratio of Sr:(Ce+Yb) of about 1:1; and (b) calcining at least a portion of the Sr—Ce—Yb—O precursor mixture to form the OCM catalyst composition, wherein the OCM catalyst composition comprises Sr—Ce—Yb—O perovskite, and one or more oxides of a metal selected from the group consisting of Sr, Ce, and Yb.

A fourteenth aspect, which is the method of the thirteenth aspect, wherein the step (a) of forming a Sr—Ce—Yb—O precursor mixture further comprises (i) solubilizing the one or more compounds comprising a Sr cation, one or more compounds comprising a Ce cation, and one or more compounds comprising a Yb cation in an aqueous medium to form a Sr—Ce—Yb—O precursor aqueous solution; and (ii) drying at least a portion of the Sr—Ce—Yb—O precursor aqueous solution to form the Sr—Ce—Yb—O precursor mixture.

A fifteenth aspect, which is the method of the fourteenth aspect, wherein the Sr—Ce—Yb—O precursor aqueous solution is dried at a temperature of equal to or greater than about 75° C.

A sixteenth aspect, which is the method of any one of the thirteenth through the fifteenth aspects, wherein the Sr—Ce—Yb—O precursor mixture is calcined at a temperature of equal to or greater than about 650° C.

A seventeenth aspect, which is the method of any one of the thirteenth through the sixteenth aspects, wherein the one or more compounds comprising a Sr cation comprises Sr nitrate, Sr oxide, Sr hydroxide, Sr chloride, Sr acetate, Sr carbonate, or combinations thereof; wherein the one or more compounds comprising a Ce cation comprises Ce nitrate, Ce oxide, Ce hydroxide, Ce chloride, Ce acetate, Ce carbonate, or combinations thereof; and wherein the one or more compounds comprising a Yb cation comprises Yb nitrate, Yb oxide, Yb hydroxide, Yb chloride, Yb acetate, Yb carbonate, or combinations thereof.

An eighteenth aspect, which is an OCM catalyst produced by the method of any one of the thirteenth through the seventeenth aspects.

A nineteenth aspect, which is a method of making an oxidative coupling of methane (OCM) catalyst composition comprising (a) forming a Sr—Ce—Yb—O precursor aqueous solution comprising Sr nitrate, Ce nitrate, and Yb nitrate, wherein the Sr—Ce—Yb—O precursor aqueous solution is characterized by a molar ratio of Sr:(Ce+Yb) of about 1:1; (b) drying at least a portion of the Sr—Ce—Yb—O precursor aqueous solution at a temperature of equal to or greater than about 75° C. to form a Sr—Ce—Yb—O precursor mixture; and (c) calcining at least a portion of the Sr—Ce—Yb—O precursor mixture at a temperature of equal to or greater than about 650° C. to form the OCM catalyst composition, wherein the OCM catalyst composition comprises a Sr—Ce—Yb—O perovskite, and one or more oxides of a metal selected from the group consisting of Sr, Ce, and Yb.

A twentieth aspect, which is an oxidative coupling of methane (OCM) catalyst composition produced by (a) solubilizing one or more compounds comprising a Sr cation, one or more compounds comprising a Ce cation, and one or more compounds comprising a Yb cation in an aqueous medium to form a Sr—Ce—Yb—O precursor aqueous solution, wherein the Sr—Ce—Yb—O precursor aqueous solution is characterized by a molar ratio of Sr:(Ce+Yb) of about 1:1; (b) drying at least a portion of the Sr—Ce—Yb—O precursor aqueous solution at a temperature of equal to or greater than about 75° C. to form the Sr—Ce—Yb—O precursor mixture; and (c) calcining at least a portion of the Sr—Ce—Yb—O precursor mixture at a temperature of equal to or greater than about 650° C. to form the OCM catalyst composition, wherein the OCM catalyst composition comprises a Sr—Ce—Yb—O perovskite, and one or more oxides of a metal selected from the group consisting of Sr, Ce, and Yb.

A twenty-first aspect, which is a method for producing olefins comprising (a) introducing a reactant mixture to a reactor comprising an oxidative coupling of methane (OCM) catalyst composition, wherein the reactant mixture comprises methane (CH₄) and oxygen (O₂), wherein the OCM catalyst composition comprises (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof; (b) allowing at least a portion of the reactant mixture to contact at least a portion of the OCM catalyst composition and react via an OCM reaction to form a product mixture comprising olefins; (c) recovering at least a portion of the product mixture from the reactor; and (d) recovering at least a portion of the olefins from the product mixture.

A twenty-second aspect, which is the method of the twenty-first aspect, wherein the OCM catalyst composition is characterized by a C₂₊ selectivity that is increased by equal to or greater than about 5%, when compared to a C₂₊ selectivity of an otherwise similar OCM catalyst composition comprising Sr—Ce—Yb—O perovskite without the one or more oxides; and wherein the OCM catalyst composition is characterized by a C₂₊ productivity that is increased by equal to or greater than about 50%, when compared to a C₂₊ productivity of an otherwise similar OCM catalyst composition comprising Sr—Ce—Yb—O perovskite without the one or more oxides.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1. An oxidative coupling of methane (OCM) catalyst composition comprising (i) Sr—Ce—Yb—O) perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof.

2. The OCM catalyst composition of claim 1, wherein the one or more oxides comprise CeO₂, CeYbO, Sr₂CeO₄, or combinations thereof.

3. The OCM catalyst composition of claim 1, wherein the single metal oxide comprises one metal cation selected from the group consisting of Sr, Ce, and Yb.

4. The OCM catalyst composition of claim 1, wherein the single metal oxide comprises CeO₂.

5. The OCM catalyst composition of claim 1, wherein the mixed metal oxide comprises two or more different metal cations, wherein each metal cation can be independently selected from the group consisting of Sr, Ce, and Yb.

6. The OCM catalyst composition of claim 1, wherein the mixed metal oxide comprises CeYbO, Sr₂CeO₄, or both CeYbO and Sr₂CeO₄.

7. The OCM catalyst composition of claim 1 having the overall general formula SrCe₍₁₋ₓ₎YbₓO_{(3-x/2)}, wherein x is from about 0.01 to about 0.99.

8. The OCM catalyst composition of claim 1 having the overall general formula Sr_{1.0}Ce_{0.9}Yb_{0.1}O_{y}, wherein y balances the oxidation states.

9. The OCM catalyst composition of claim 1 comprising (i) from about 10.0 wt. % to about 90.0 wt. % Sr—Ce—Yb—O perovskite; and (ii) from about 10.0 wt. % to about 90.0 wt. % one or more oxides.

10. The OCM catalyst composition of claim 1 further comprising a support, wherein at least a portion of the OCM catalyst composition contacts, coats, is embedded in, is supported by, and/or is distributed throughout at least a portion of the support; wherein the support comprises MgO, Al₂O₃, SiO₂, ZrO₂, or combinations thereof and wherein the support is in the form of particles, pellets, monoliths, foams, honeycombs, or combinations thereof.

11. The OCM catalyst composition of claim 1, wherein the OCM catalyst composition is characterized by a C₂₊ selectivity that is increased by equal to or greater than about 5%, when compared to a C₂₊ selectivity of an otherwise similar OCM catalyst composition comprising Sr—Ce—Yb—O perovskite without the one or more oxides.

12. The OCM catalyst composition of claim 1, wherein the OCM catalyst composition is characterized by a C₂₊ productivity that is increased by equal to or greater than about 50%, when compared to a C₂₊ productivity of an otherwise similar OCM catalyst composition comprising Sr—Ce—Yb—O perovskite without the one or more oxides.

13. A method of making an oxidative coupling of methane (OCM) catalyst composition comprising:
(a) forming a Sr—Ce—Yb—O precursor mixture, wherein the Sr—Ce—Yb—O precursor mixture comprises one or more compounds comprising a Sr cation, one or more compounds comprising a Ce cation, and one or more compounds comprising a Yb cation, and wherein the Sr—Ce—Yb—O precursor mixture is characterized by a molar ratio of Sr:(Ce+Yb) of about 1:1; and
(b) calcining at least a portion of the Sr—Ce—Yb—O precursor mixture to form the OCM catalyst composition, wherein the OCM catalyst composition comprises Sr—Ce—Yb—O perovskite, and one or more oxides of a metal selected from the group consisting of Sr, Ce, and Yb.

14. The method of claim 13, wherein the step (a) of forming a Sr—Ce—Yb—O precursor mixture further comprises (i) solubilizing the one or more compounds comprising a Sr cation, one or more compounds comprising a Ce cation, and one or more compounds comprising a Yb cation in an aqueous medium to form a Sr—Ce—Yb—O precursor aqueous solution; and (ii) drying at least a portion of the Sr—Ce—Yb—O precursor aqueous solution to form the Sr—Ce—Yb—O precursor mixture.

15. The method of claim 14, wherein the Sr—Ce—Yb—O precursor aqueous solution is dried at a temperature of equal to or greater than about 75° C.

16. The method of claim 13, wherein the Sr—Ce—Yb—O precursor mixture is calcined at a temperature of equal to or greater than about 650° C.

17. The method of claim 13, wherein the one or more compounds comprising a Sr cation comprises Sr nitrate, Sr oxide, Sr hydroxide, Sr chloride, Sr acetate, Sr carbonate, or combinations thereof; wherein the one or more compounds comprising a Ce cation comprises Ce nitrate, Ce oxide, Ce hydroxide, Ce chloride, Ce acetate, Ce carbonate, or combinations thereof; and wherein the one or more compounds comprising a Yb cation comprises Yb nitrate, Yb oxide, Yb hydroxide, Yb chloride, Yb acetate, Yb carbonate, or combinations thereof.

18. An OCM catalyst produced by the method of claim 13.

19. A method for producing olefins comprising:
(a) introducing a reactant mixture to a reactor comprising an oxidative coupling of methane (OCM) catalyst composition, wherein the reactant mixture comprises methane (CH₄) and oxygen (O₂), wherein the OCM catalyst composition comprises (i) Sr—Ce—Yb—O perovskite; and (ii) one or more oxides of a metal selected from the group consisting of strontium (Sr), cerium (Ce), and ytterbium (Yb), wherein the one or more oxides comprises a single metal oxide, mixtures of single metal oxides, a mixed metal oxide, mixtures of mixed metal oxides, mixtures of single metal oxides and mixed metal oxides, or combinations thereof;
(b) allowing at least a portion of the reactant mixture to contact at least a portion of the OCM catalyst composition and react via an OCM reaction to form a product mixture comprising olefins;
(c) recovering at least a portion of the product mixture from the reactor; and
(d) recovering at least a portion of the olefins from the product mixture.

20. The method of claim 19, wherein the OCM catalyst composition is characterized by a C₂₊ selectivity that is increased by equal to or greater than about 5%, when compared to a C₂₊ selectivity of an otherwise similar OCM catalyst composition comprising Sr—Ce—Yb—O perovskite without the one or more oxides; and wherein the OCM catalyst composition is characterized by a C₂₊ productivity that is increased by equal to or greater than about 50%, when compared to a C₂₊ productivity of an otherwise similar OCM catalyst composition comprising Sr—Ce—Yb—O perovskite without the one or more oxides.
